(19) United States
(12) Patent Application Publication
(10) Pub. No.: US 2026/0167937 A2
(43) Pub. Date: Jun. 18, 2026

(54) FLOCCULATION AAV PURIFICATION

(71) Applicants: Bruno Figueroa, New York, NY (US); Qingxuan Li, Malden, MA (US); Junfen Ma, Cambridge, MA (US); Yuanli Song, Cambridge, MA (US); Mahsa Hadidi, Cambridge, MA (US)

(72) Inventors: Bruno Figueroa, Andover, MA (US); Qingxuan Li, Malden, MA (US); Junfen Ma, Cambridge, MA (US); Yuanli Song, Cambridge, MA (US); Mahsa Hadidi, Cambridge, MA (US)

(73) Assignee: Genzyme Corporation, Cambridge, MA (US)

(21) Appl. No.: 18/511,501

(22) Filed: Nov. 16, 2023

Related U.S. Application Data
(60) Provisional application No. 63/425,998, filed on Nov. 16, 2022.

(51) Int. Cl. C12N 7/00

(52) U.S. Cl. CPC C12N 7/00; C12N 2750/14151

(57) ABSTRACT

The disclosure provides methods of purifying adeno-associated virus (AAV) particles by introducing a flocculant step into a large scale a manufacturing process, thereby improving AAV particle purity and yield.

### RELATED APPLICATIONS

This application claims the benefit under 35 § 119 (e) of U.S. Provisional Application No. 63/425,998, filed on Nov. 16, 2022, entitled “FLOCCULATION AAV PURIFICATION,” the contents of which are hereby incorporated by reference in their entirety.

### FIELD OF THE INVENTION

The invention relates to methods for purifying recombinant AAV particles for use in gene therapy.

### BACKGROUND OF THE INVENTION

During typical gene therapy manufacturing, viral vectors are produced in cell cultures and isolated from harvested culture cells in a process that involves a cell lysis step. Isolated viral vector preparations contain impurities from the manufacturing process, including cellular material released during cell lysis. The impurities can cause instability of the viral vectors and also contribute a significant burden to the downstream purification steps.

Therefore, there is a need to improve current manufacturing processes for viral vectors used in gene therapy.

### BRIEF SUMMARY OF THE INVENTION

The application provides methods and compositions for purifying recombinant adeno-associated virus (rAAV) particles from cell culture. In some aspects, methods and compositions are useful for large scale manufacturing of rAAV for use in gene therapy and can increase the purity and stability of the rAAV compositions.

In some embodiments, rAAV particles are isolated from a cell culture comprising the rAAV particles using a process comprising: a) contacting an rAAV preparation obtained from a cell culture with an acid glycine solution under conditions sufficient to promote flocculation of cellular material that is present in the rAAV preparation; and b) separating rAAV particles from flocculated cellular material.

In some embodiments, the rAAV preparation is a cell culture harvest comprising rAAV particles. In some embodiments, the rAAV preparation is a cell culture lysate obtained from the cell culture harvest. In some embodiments, the lysate is obtained using a chemical lysis technique. In some embodiments, a nucleic acid degradation technique is used to obtain the rAAV preparation. For example, in some embodiments, an rAAV preparation is obtained in a process that includes contacting a cell lysate containing rAAV particles with a nuclease.

In some embodiments, the pH of the acid glycine solution that is used to flocculate cellular material is below 4. In some embodiments, the pH of the acid glycine solution is about 2.5. In some embodiments, a 1-3 M acid glycine solution is added to an rAAV preparation. In some embodiments, a 2M acid glycine solution at pH 2.5 is added to the rAAV preparation.

In some embodiments, the acid glycine solution is added to the rAAV preparation at a volume of 5-10%. In some embodiments, the acid glycine solution is added to the rAAV preparation at a volume of 8%. In some embodiments, the volume of acid glycine solution is added to the rAAV preparation within a period of 10 minutes. In some embodiments, the volume of acid glycine solution is added to the rAAV preparation within a period of 5 minutes.

In some embodiments, the rAAV preparation is mixed with the added acid glycine solution using an agitation speed of 30-150 RPM. In some embodiments, the agitation speed is 100 RPM.

In some embodiments, the volume of the rAAV preparation is 2-500L. In some embodiments, the volume of the AAV preparation is 5L, 50L, or 500L.

In some embodiments, the volume of the rAAV preparation is about 5L and the agitation speed is about 90-110 RPM, for example about 100 RPM. In some embodiments, the volume of the rAAV preparation is about 50L and the agitation speed is about 50-75 RPM, for example about 63 RPM. In some embodiments, the volume of the AAV preparation is about 500L and the agitation speed is about 30-50 RPM, for example about 42 RPM.

In some embodiments, the product of a) has a pH of 3-5 after addition of the glycine (e.g., after addition of a volume of 5-10%, for example 8%, of 2M glycine at pH 2.5). In some embodiments, the pH of the product of a) is around (e.g., about) 4.

In some embodiments, the mixture of the rAAV preparation and acid glycine solution of a) is held static in a vessel for 10-60 minutes (e.g., at room temperature) to promote flocculation of the cellular material. In some embodiments, the mixture is held static for 15-45 minutes. In some embodiments, the mixture is held static for about 30 minutes. In some embodiments, the flocculated material from a) is resuspended prior to separating rAAV particles from flocculated cellular material.

In some embodiments, the product of a) is clarified. In some embodiments, a resuspended product of a) is clarified. In some embodiments, the clarification is via filtration. In some embodiments, the filtration is depth filtration.

In some embodiments, glycine is the only pH-reducing agent that is used to flocculate the cellular material. However, in some embodiments, an alternative or additional flocculation agent can be used. In some embodiments, the alternative or additional flocculation agent is a pH-reducing agent or a cationic polymer. In some embodiments, the alternative or additional pH-reducing agent can include citric acid, phosphoric acid, and/or caprylic acid. In some embodiments, a cationic polymer is polyethylenimine (PEI) or polydiallyldimethylammonium chloride (pDADMAC).

In some embodiments, a lysis agent, for example a detergent, can be used along with the flocculation agent.

In some embodiments, the rAAV particles comprise capsid proteins of an AAV1, AAV2, AAV3, AAV4, AAV5, AAV6, AAV7, AAV8, AAV9, AAV10, AAV11, or AAV12 serotype, or variants thereof. In some embodiments, the rAAV particles are rAAV9 particles.

In some embodiments, the rAAV particles comprise a recombinant nucleic acid (e.g., a recombinant AAV genome) including a recombinant gene of interest flanked by AAV inverted terminal repeats (ITRs). In some embodiments, the gene of interest encodes a therapeutic RNA or protein.

In some embodiments, rAAV particles are further purified, for example using one or more affinity, ion exchange chromatography, and/or hydrophobic interaction chromatography steps, e.g., after clarification of an rAAV preparation.

In some embodiments, rAAV particles (e.g., after one or more purification steps) are added to a pharmaceutically acceptable solution.

The application also provides compositions comprising rAAV particles, and methods of administering the rAAV particles to a subject (e.g., a human subject having a condition that the therapeutic RNA and/or protein can help treat).

These and other embodiments are described in the following Detailed Description and Examples, along with the Figures.

### BRIEF DESCRIPTION OF DRAWINGS

FIGS. 1A and 1B illustrate non-limiting embodiments of harvest procedures using flocculation at a manufacturing process scale. FIG. 1A shows a schematic of a flocculation procedure. FIG. 1B illustrates a non-limiting embodiment of a flocculation step incorporated into a large scale rAAV manufacturing process.

FIGS. 2A and 2B show flocculant screening graphs. FIG. 2A shows non-limiting examples of host cell protein (HCP) reduction after flocculation under different flocculant conditions. FIG. 2B shows non-limiting examples of rAAV titer recovery post-flocculation.

FIGS. 3A and 3B show non-limiting examples of depth filtration performance with and without flocculation. The HCP reduction post depth filtration is illustrated in FIG. 3A, and the improvement of throughput after flocculation is illustrated in FIG. 3B.

FIGS. 4A and 4B show ultrafiltration/diafiltration (UFDF) performance with and without flocculation. FIG. 4A shows the UFDF performance with the flocculated material has double flux with no detectable flux decay, compared to the one with non-flocculated material. FIG. 4B shows the operational time with flocculated material is two times faster than the time with non-flocculated material.

FIGS. 5A-5C show the effect of flocculation on capture chromatography performance. FIGS. 5A and 5B show capture chromatography performance with (A) and without (B) flocculation. FIG. 5C shows host cell DNA reduction in the affinity eluate with and without flocculation.

FIGS. 6A and 6B show polishing chromatography performance with (A) and without (B) flocculation. The averaged product sizes before and after dilution are shown below to indicate the product stability.

FIGS. 7A and 7B show full capsid enrichment on polishing chromatography with the flocculated (A) and non-flocculated material (B). The highlighted area in the chromatogram shows the enriched region. The table below shows the full capsid enrichment percentage after polishing step.

FIG. 8 shows product stability after UFDF flocculated (triangle) or non-flocculated (circle) material.

FIG. 9 shows the recovery of AAV from the producer cell line platform after flocculation.

FIG. 10 shows host cell DNA reduction post flocculation for a non-limiting producer cell line platform.

FIGS. 11A and 11B show affinity resin cycling numbers for purifying non-flocculated (A) and flocculated material (B).

FIG. 12 shows that AAV product becomes more stable after flocculation treatment at harvest step.

FIG. 13 shows product stability during low pH hold. No loss of AAV titer was observed upon low pH flocculation.

FIG. 14 shows that flocculated material shows no turbidity increase after heat inactivation.

### DETAILED DESCRIPTION OF THE INVENTION

Recombinant adeno-associated virus (rAAV) vectors are useful in gene therapy to deliver therapeutic genes to patient cells and tissue. An rAAV particle typically comprises a recombinant nucleic acid encapsidated within AAV capsid proteins to form an rAAV particle that can be administered to a subject. The recombinant nucleic (e.g., recombinant AAV genome) acid typically includes a heterologous gene of interest (e.g., encoding a therapeutic nucleic acid and/or protein) flanked by AAV inverted terminal repeat (ITR) sequences. The AAV capsid proteins can be naturally occurring capsids of different AAV serotypes. For example, different AAV serotypes have different tissue tropisms and can be used to target different tissue types and associated diseases. In some embodiments, the AAV capsid proteins include one or more amino acid substitutions relative to naturally occurring capsid proteins.

Different manufacturing techniques can be used to produce rAAV particles. Typically, rAAV particles are assembled in host cells in culture (e.g., in a bioreactor or other cell culture vessel). One or more nucleic acids encoding the recombinant AAV genome, AAV capsid proteins, and/or one or more Rep and helper genes are expressed in the host cell. The host cell is grown in culture (e.g., in a suspension culture, or on plates). The assembled rAAV is then isolated from the cell culture. The host cell can be a mammalian cell, an insect cell or other cell type. In some embodiments, a host cell is a producer cell.

Isolated rAAV preparations made from large scale culture processes typically contain contaminating material, including host cell material, that can interfere with the purification process and/or destabilize purified rAAV.

In some embodiments, aspects of the application relate to the incorporation of a flocculation step in an rAAV manufacturing procedure. In some embodiments, an acid solution is added to an rAAV preparation under conditions that promote effective removal of host cell material (e.g., host cell proteins). In some embodiments, the acid solution is mixed with a cell preparation in an amount sufficient and within a time sufficient for effective removal of host cell material. In some embodiments, the cell preparation comprises a plurality of cells for producing rAAV. In some embodiments, the cell preparation comprises a plurality of triple-transfected cells. In some embodiments, the cell preparation comprises a plurality of producer cells. In some embodiments, the acid solution is mixed with a cell preparation after cell lysis. In some embodiments, the acid solution is not a triprotic acid solution. In some embodiments, the acid solution is an acid glycine solution.

In some embodiments, a cell preparation is at a density suitable for rAAV harvest. In some embodiments, the cell preparation has a density of about 0.5-12×10⁶ cells/mL. In some embodiments, the cell preparation has density of about 0.5-2, about 2-4, about 4-6, about 6-8, about 8-10, or about 10-12×10⁶ cells/mL. In some embodiments, the cell preparation has a density of 0.5-1, 2-3, 3-4, 4-5, 5-6, 6-7. 7-8. 8-9. 9-10, or 11-12×10⁶ cells/mL.

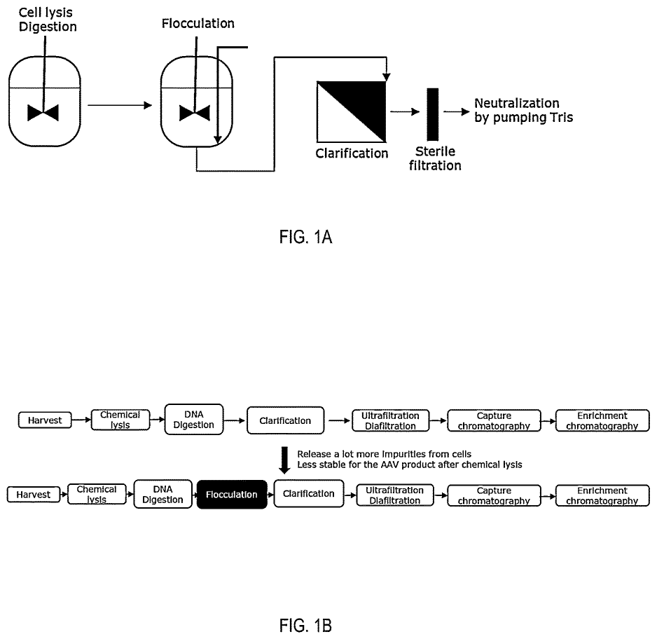

FIG. 1A illustrates a non-limiting example of a procedure for adding a flocculating agent (e.g., an acid solution to a cell preparation comprising rAAV particles. In some embodiments, the flocculating agent is an acid glycine solution. In some embodiments, the flocculating agent is a solution at a pH of 4 or below, a solution of a pH of 3 or below, a solution at a pH of 2 or below. In some embodiments, the flocculating agent is a solution at a pH about 4, about 3.5, about 3, about 2.5, about 2, about 1.5 or about 1. In some embodiments, the flocculating agent can be added to a vessel (e.g., a bioreactor) that includes a mixing device (e.g., an impeller). In some embodiments, the appropriate speed of the impeller can be determined using one or more equations (1), (2), and/or (3) set out in Example 1.

In some embodiments, a cell preparation comprises a cell culture. In some embodiments, a cell preparation comprises a resuspended cell pellet. In some embodiments, a cell preparation comprises a plurality of cells for producing rAAV. In some embodiments, the cell preparation comprises a plurality of triple-transfected cells. In some embodiments, the cell preparation comprises a plurality of producer cells. In some embodiments, the acid solution is mixed with a cell preparation after cell lysis. In some embodiments, a cell preparation is a cell harvest.

FIG. 1B illustrates non-limiting examples of a procedure for isolating rAAV particles from a cell culture. In some embodiments, a flocculation step (e.g., using an acid glycine solution) is incorporated after the cell lysis and nuclease steps and before subsequent clarification and additional purification steps as illustrated in FIG. 1B. However, in some embodiments, a flocculation step can be incorporated before cell lysis and nuclease steps, in between cell lysis and nuclease steps, at the same time as a cell lysis and/or nuclease steps, and/or as a substitute for cell lysis and/or nuclease addition. In some embodiments, cell lysis comprises mechanical lysis, liquid homogenization, sonication, freeze/thaw cycles, or chemical lysis. In some embodiments, chemical lysis conditions comprise treatment with detergents, such as Tween 20 or Triton X-100. In some embodiments, a nuclease is an endonuclease. In some embodiments, the nuclease is or comprises Benzonase® (Merck, endonuclease derived from Serratia marcesens, optionally expressed in Escherichia Coli). In some embodiments, the nuclease is M-SAN HQ (nuclease; ArcticZymes)

In some embodiments, methods of flocculating cellular material are adapted for large scale culture and isolation processes and provide surprising improvements over existing methods. In some embodiments, a large scale culture comprises a culture over 1L, over 10L, over 25L, over 50L, over 100L, over 250L, or over 500L. In some embodiments, a large scale culture comprises 1-10L, 10-25L, 25-50L, 50-100L, 100-500, or 500-1000L. In some embodiments, subsequent processing steps are significantly more efficient (e.g., shorter processing times and higher yield). In some embodiments, the resulting rAAV products are more stable. For example, in some embodiments, introduction of a flocculation process described in this application at the process scale efficiently remove impurities and results in a 4-fold to 5-fold host cell protein (HCP) reduction for downstream purification process.

In some embodiments, methods that are useful at a process scale comprise purifying recombinant adeno-associated virus (rAAV) particles from a cell culture comprising the rAAV particles by contacting an rAAV preparation obtained from a cell culture with an acid solution (e.g., an acid glycine solution, a citric acid solution (also referred to as “citrate acid”), a caprylic acid solution) under conditions sufficient to promote flocculation of cellular material that is present in the rAAV preparation prior to subsequent purification of the rAAV. In some embodiments, the rAAV preparation is a cell culture harvest comprising rAAV particles. In some embodiments, the rAAV preparation is a cell culture lysate (e.g., a chemical lysate) comprising rAAV particles. In some embodiments, the rAAV preparation is contacted with a nuclease (e.g., after lysis and before flocculation). However, in some embodiments, no nuclease is added prior to flocculation.

In some embodiments, the pH of the acid solution is below 4 (e.g., about 2.5). In some embodiments, sufficient acid is added to reduce the pH of the rAAV preparation to around (e.g., about) 2-4, around 3-4, around 3-5, around 4-5, around 2.5-3.5, around 2.5-4.5, around 3.5-5.5 (e.g., around pH 4). In some embodiments, the acid solution is about a 0.5M solution, about a 1M solution, about a 2M solution, about a 3M solution, about a 4M solution, about a 5M solution, about a 6M solution, about a 7M solution, about an 8M solution, about a 9M solution, or about a 10M solution. In some embodiments, a 2M acid solution at pH 2.5 is added to the rAAV preparation. In some embodiments, the pH of the rAAV preparation is adjusted to be about pH 4 by addition of an acid solution (e.g., a 2M acid glycine solution).

In some embodiments, the acid solution is an acid glycine solution. In some embodiments, the pH of the acid glycine solution is below 4 (e.g., about 2.5). In some embodiments, sufficient acid glycine is added to reduce the pH of the rAAV preparation to around (e.g., about) 2-4, around 3-4, around 3-5, around 4-5, around 2.5-3.5, around 2.5-4.5, around 3.5-5.5 (e.g., around pH 4). In some embodiments, the acid glycine solution is about a 1M solution, about a 2M solution, about a 3M solution, about a 4M solution, about a 5M solution, about a 6M solution, about a 7M solution, about an 8M solution, about a 9M solution, or about a 10M solution. In some embodiments, a 2M acid glycine solution at pH 2.5 is added to the rAAV preparation.

In some embodiments, the acid solution is a citric acid solution. In some embodiments, the pH of the citric acid solution is below 4 (e.g., about 2.5). In some embodiments, sufficient citric acid is added to reduce the pH of the rAAV preparation to around (e.g., about) 2-4, around 3-4, around 3-5, around 4-5, around 2.5-3.5, around 2.5-4.5, around 3.5-5.5 (e.g., around pH 4). In some embodiments, the citric acid solution is about a 1M solution, about a 2M solution, about a 3M solution, about a 4M solution, about a 5M solution, about a 6M solution, about a 7M solution, about an 8M solution, about a 9M solution, or about a 10M solution. In some embodiments, a 2M citric acid solution at pH 2.5 is added to the rAAV preparation.

In some embodiments, the acid solution (e.g., acid glycine solution) is added to the rAAV preparation at a volume of 5-10% (e.g., around 8%). In some embodiments, the acid solution is added to the rAAV preparation at a volume of 1-10%, 1-5%, 2-9%, 3-8%, 4-7%, 5-9%, or 4-8%. In some embodiments, the acid solution is added to the rAAV preparation over a period of about 10 minutes (e.g., within a period of around (e.g., about) 5 minutes). In some embodiments, the rAAV preparation is mixed with the added acid solution using an agitation speed of around (e.g., about) 30-150 RPM. In some embodiments, the rAAV preparation is mixed with the added acid solution using an agitation speed of around (e.g., about) 50-150 RPM. In some embodiments, an agitation speed of around (e.g., about) 90-110 RPM (e.g., about 100 RPM) is used for approximately 5L of the rAAV preparation. In some embodiments, an agitation speed of around (e.g., about) 30-200 RPM or 90-200 RPM is used for approximately 5L of the rAAV preparation In some embodiments, an agitation speed of around (e.g., about) 50-75 RPM (e.g., about 63 RPM) is used for approximately 50L of the rAAV preparation. In some embodiments, an agitation speed of around (e.g., about) 50-100 or 50-150 RPM is used for approximately 50L of the rAAV preparation. In some embodiments, an agitation speed of around 30-50 RPM (e.g., about 42 RPM) is used for approximately 500L of the rAAV preparation. In some embodiments, an agitation speed of around 30-100 is used for approximately 500L of the rAAV preparation. In some embodiments, the agitation speed is adjusted to achieve a power/volume (P/V) ratio of around (e.g., about) 2-5. In some embodiments, the agitation speed is adjusted to achieve a P/V ratio of around 4.7. In some embodiments, the agitation speed is adjusted to achieve a P/V ratio of around 3.1. In some embodiments, the mixture of the rAAV preparation and acid solution (e.g., acid glycine solution) of is held static in a vessel for 10-60 minutes (e.g., at room temperature) to promote flocculation of the cellular material prior to subsequent purification steps. In some embodiments, the mixture is held static for 15-45 minutes (e.g., the hold time is 15-45 minutes). In some embodiments, the mixture is held static for about 30 minutes (e.g., the hold time is 30 minutes). In some embodiments, the mixture is held static for up to 10 hour, up to 12 hours, up to 14 hours, up to 16 hours, up to 18 hours, up to 20 hours, up to 22 hours, or up to 24 hours; in some embodiments, the mixture is held static for between 30 minutes and 4 hours, between 30 minutes and 10 hours, between 10 minutes and 5 hours, between 20 minutes and 6 hours, between 10 minutes and 4 hours, or between 1 hour and 4 hours. In some embodiments, the mixture of the rAAV preparation and acid solution (e.g., acid glycine solution) of is agitated slowly (e.g., at 30-150 rpm) in a vessel for 10-60 minutes (e.g., at room temperature) to promote flocculation of the cellular material prior to subsequent purification steps. In some embodiments, the mixture is agitated slowly (e.g., at 30-150 rpm) for 15-45 minutes. In some embodiments, the mixture is agitated slowly (e.g., at 30-150 rpm) for about 30 minutes. In some embodiments, the mixture is agitated slowly (e.g., at 30-150 rpm) for up to 10 hours; in some embodiments, the mixture is agitated slowly (e.g., at 30-150 rpm) for between 30 minutes and 4 hours, between 30 minutes and 10 hours, between 10 minutes and 5 hours, between 20 minutes and 6 hours, between 10 minutes and 4 hours, or between 1 hour and 4 hours. In some embodiments, the flocculated material from is resuspended prior to subsequent purification (e.g., prior to one or more clarification steps). Accordingly, in some embodiments, the flocculated mixture is clarified without an intervening resuspension. In some embodiments, the flocculate mixture is resuspended prior to clarification. In some embodiments, the clarification is via filtration. In some embodiments, the filtration is depth filtration.

In some embodiments, the method is carried out at a room temperature. In some embodiments the method is carried out at 10-40 C, for example 15-35 C, 15-20 C, 20-25 C, or 25-30 C.

In some embodiments, a method comprises contacting the rAAV preparation with a flocculation agent (e.g., acid glycine). In some embodiments, a method comprises contacting the rAAV preparation with glycine. In some embodiments, a method comprises contacting the rAAV preparation with an alternative or additional flocculation agent, for example, a cationic polymer, for example polyethylenimine (PEI) or Polydiallyldimethylammonium chloride (pDADMAC), etc., and/or an alternative or additional pH-reducing agent, for example citric acid, phosphoric acid, and/or caprylic acid, and/or an alternative or additional lysis agent, for example, a detergent. In some embodiments, the detergent is Triton, PS20 (tween 20), or other detergent.

The disclosure also provides compositions comprising AAV particles produced by the methods as described herein. In some embodiments, an rAAV preparation after flocculation but before subsequent purification steps is more stable than a corresponding preparation without flocculation. In some embodiments, a post-flocculation rAAV preparation can be held (e.g., for up to 2 weeks or more). In some embodiments, one or more post-flocculation rAAV preparations can be held, e.g., for 1-2 weeks or more, and then combined for subsequent purification steps.

### Recombinant AAVs

Naturally occurring AAV capsid proteins can be used to produce rAAVs for gene therapy. Different naturally occurring AAVs have different characteristics (including for example different tissue tropisms) and can be used for different indications. AAVs are highly prevalent within the human population (see Gao, G., et al., Clades of Adeno-associated viruses are widely disseminated in human tissues J Virol. 2004. 78 (12): p. 6381-8, and Boutin. S., et al., Prevalence of serum IgG and neutralizing factors against adeno-associated virus (AAV) types 1, 2, 5, 6, 8, and 9 in the healthy population, implications forgone therapy using AAV vectors. Hum Gene Ther. 2010. 21 (6): p. 704-12) and are useful as viral vectors. Many serotypes exist, each with different tropism for tissue types (see Zincarelli, C., et al., Analysis of AAV serotypes 1-9 mediated gene expression and tropism in mice after systemic injection. Mol Ther, 2008. 16 (6): p. 1073-80), which allows specific tissues to be preferentially targeted with appropriate pseudotyping. Some serotypes, such as serotypes 8, 9, and rh10, transduce the mammalian body. See Zincarelli, C., et al. Analysis of AAV serotypes 1-9 mediated gene expression and tropism in mice after systemic injection. Mol Ther, 2008. 16 (6): p. 1073-80, Inagaki, K., et al., Robust systemic transduction with AAV9 vectors in mice: efficient global cardiac gene transfer superior to that of AAV8. Mol Ther, 2006. 14 (1): p. 45-53, Keeler, A. M., et al., Long-term correction of very long-chain acyl-coA dehydrogenase deficiency in mice using AAV9 gene therapy. Mol Ther, 2012. 20 (6): p. 1131-8, Gray, S. J., et al., Preclinical differences of intravascular AAV9 delivery to neurons and glia: a comparative study of adult mice and nonhuman primates. Mol Ther, 2011. 19 (6): p. 1058-69, Okada, H., et al., Robust Long-term Transduction of Common Marmoset Neuromuscular Tissue With rAAV1 and rAAV9. Mol Ther Nucleic Acids, 2013. 2: p. e95, and Foust, K. D., et al., Intravascular AAV9 preferentially targets neonatal neurons and adult astrocytes. Nat Biotechnol, 2009. 27 (1): p. 59-65. AAV9 has been demonstrated to cross the blood-brain barrier (see Foust, K. D., et al., Intravascular AAV9 preferentially targets neonatal neurons and adult astrocytes. Nat Biotechnol, 2009. 27 (1): p. 59-65, and Rahim, A. A., et al., Intravenous administration of AAV2/9 to the fetal and neonatal mouse leads to differential targeting of CNS cell types and extensive transduction of the nervous system. FASEB J, 2011. 25 (10): p. 3505-18) that is inaccessible to many viral vectors and biologics. Certain AAVs have a payload of 4.7-5.0 kb (including viral inverted terminal repeats (ITRs), which are required in cis for viral packaging). See Wu, Z., H. Yang, and P. Colosi, Effect of genome size on AAV vector packaging. Mol Ther, 2010. 18 (1): p. 80-6 and Dong, J. Y., P. D. Fan, and R. A. Frizzell, Quantitative analysis of the packaging capacity of recombinant adeno-associated virus. Hum Gene Ther, 1996. 7 (17): p. 2101-12.

In some embodiments, rAAVs can include one or more variant AAV capsid proteins have one or more amino acid substitutions relative to a naturally occurring AAV capsid protein.

Accordingly, in some embodiments, the rAAV particles comprise AAV1, AAV2, AAV3, AAV4, AAV5, AAV6, AAV7, AAV8, AAV9, AAV10, AAV11, or AAV12 capsid proteins, or amino acid sequence variants thereof. In some embodiments, the rAAV particles comprise a hybrid capsid protein derived from any combination of AAV1, AAV2, AAV3, AAV4, AAV5, AAV6, AAV7, AAV8, AAV9, AAV10, AAV11, or AAV12 capsid proteins.

In some embodiments, the methods described herein are beneficial to the manufacturing process. These benefits include, but are not limited to, more than 10-fold host cell impurity reduction without use of endonuclease, efficient filtration without flux decay, and 5-times higher affinity resin lifecycles due to the low impurity in the load material. Furthermore, with less interference from impurities, the subtle charge difference between three kinds of capsids enables a higher resolution and better enrichment of full capsids. By controlling impurities level at upstream feed stream, rAAV viral vectors demonstrate improved stability with minimal aggregation at low conductivity, which further improves process recovery. With cleaner feed stream, the downstream intermediates achieve more than 10-times lower turbidity values with maintained rAAV titers, enabling easy filtration and manufacturing robustness. Flocculation has been successfully demonstrated as an innovative rAAV manufacturing technology for multiple AAV serotypes. The implementation in rAAV process platform gains not only superior product quality, significant benefits to downstream recovery, but also a major cost of saving in rAAV manufacturing.

These and other aspects are illustrated by the following non-limiting examples.

### EXAMPLES

### Example 1

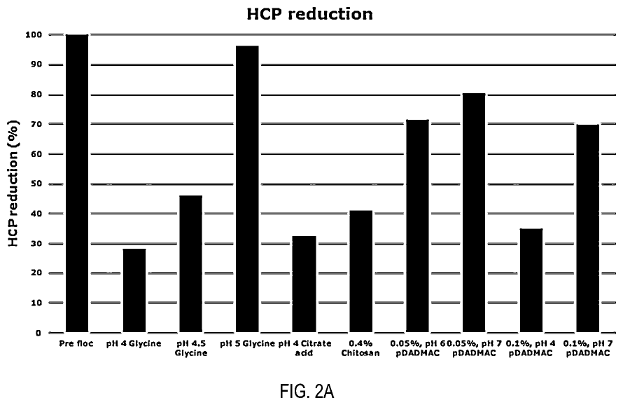

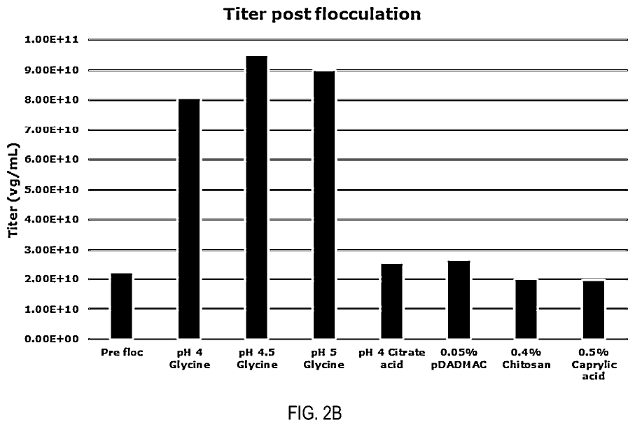

As shown in FIGS. 1A and 1B, 2M glycine acid at pH 2.5 was used to bring the pH of the cell culture harvest down to pH 4. After cell lysis and digestion, the impeller speed was reduced to match the power/volume ratio of 3.1 in some experiments and 4.7 in some experiments. Similar calculations of power/volume ratio (e.g., 2-5) are expected to behave similarly. The cell lysis and digestion steps were carried out in the context of purification of AAV generated using a triple transfection method but are optional for AAV generated from a producer cell line. The flocculation buffer was pumped into bioreactor through the dip tube near the impeller. There were two steps of acid addition with a total target volume of 8% of the cell culture harvest volume. 80% of the target volume was first added with a minute of holding time for pH reading and the rest of the glycine was pumped until a target of pH 4 was reached. The pumping rate was calculated by limiting the acid addition to a 10-minute addition period. When the pH was reached, the flocculated material was held static (e.g., without agitation) for 30 minutes to allow large particle formation. The two phases (precipitate and supernatant) were re-mixed and loaded onto a Clarisolve depth filter. The filtrate was neutralized by adding 5% V/V 2M Tris buffer right after depth filtration.

The flocculation efficiency can be evaluated using the following equation (1):

$$C r = f ( {C}_{0} , {t}_{F} , {C}_{F} , N , \frac{P}{V} , pH , T ) . \qquad ( 1 )$$

Where C₀ is the initial impurity level, and the impurity level is assumed the same under same cell density and same lysis condition, t_{F} is the flocculation time to precipitate impurities and form large particles, C_{F} is the flocculent dosage, N is impeller agitation speed which can impact the mixing efficiency during flocculant addition and also the size of the precipitates, P/V is the power input, approximate to the average turbulent kinetic energy dissipation εₐᵥₑ, pH is the target pH in the bioreactor after flocculation and T is the temperature in the bioreactor. Cr is the impurity level after flocculation, which can be the criteria to evaluate flocculation efficiency. As shown in equation (2), the P/V ratio is proportional to the impeller type, configuration, spacing (Np), impeller speed (N), liquid density (p) and impeller diameter (D).

$$\frac{P}{V} = \frac{{N}_{P} ρ {N}^{3} {D}^{5}}{V} , \qquad ( 2 )$$

$${N}_{L} = {{N}_{S} ( \frac{{D}_{S}}{{D}_{L}} )}^{2 / 3} , \qquad ( 3 )$$

To implement flocculation at a process scale, the scale-up rule was based on the same flocculation efficiency. Given a target pH and T, the flocculation efficiency was based on a constant P/V ratio, flocculation time (t_{F}) and flocculation dosage (C_{F}/C₀). As shown in equation (2). the agitation speed was determined by the geometry of the bioreactor. Given the impeller diameter in the large scale (DL) and small scale (DS), the agitation speed in the large-scale NL was determined by equation (3) to maintain the same flocculation efficiency during scale up.

### Example 2

An AAV purification process was developed to purify AAV particles from cell culture and to enrich the AAV preparation for full AAV particles (e.g., containing a recombinant AAV genome) relative to empty AAV particles (e.g., containing capsid proteins but no encapsulated nucleic acid).

An updated purification process was developed that introduced a flocculation, after DNA digestion, using 2M Glycine acid pH 2.5 as a flocculant buffer to lower the post-lysis harvest to pH 4. In some embodiments, the flocculation procedure involved developing a target agitation speed, a target pump rate at which the acid was pumped in, and/or reaching the target pH and holding the pH for target hold time. This process was developed using AAV9 as an example. This updated purification process was surprisingly effective. It was characterized by several improvements, including: a 4 to 5-fold HCP/DNA reduction at the harvest step, higher throughput on clarification, and a more stable and higher yield AAV product.

As shown in FIGS. 2A and 2B, four different acids (citrate acid, phosphoric acid, glycine and caprylic acid), and two different cationic polymers, polyethylenimine (PEI) and polydiallyldimethylammonium chloride (pDADMAC) were used as the flocculant during harvest, the titer and host cell protein reduction after flocculation were studied after using different flocculant. The HCP level showed that pH 4 was more effective than pH 4.5 and pH 5, and provided a 4-5 fold HCP reduction (FIG. 2A). Citrate acid and pDADMAC also provided detectable levels of HCP reduction. According to the titer post flocculation in FIG. 2B, there was no obvious titer loss across multiple flocculants. Higher titer was observed when using glycine acid to flocculate the cell harvest.

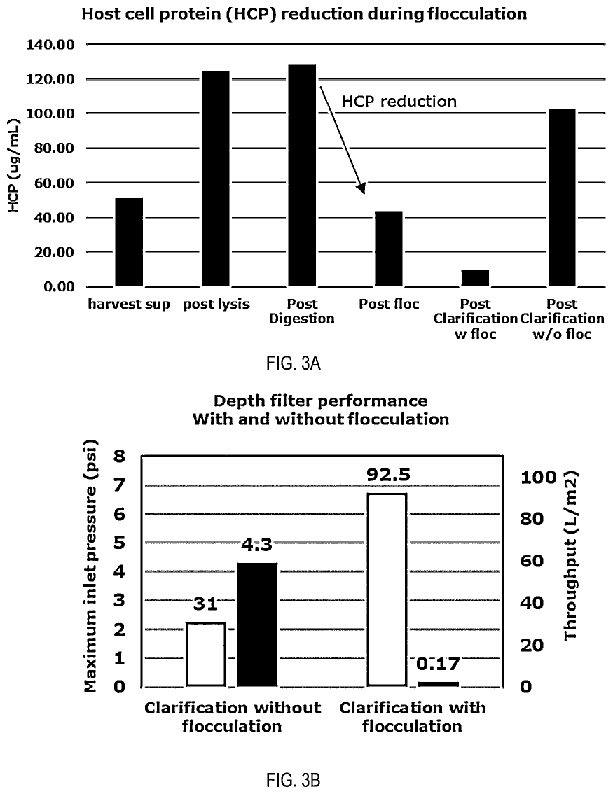

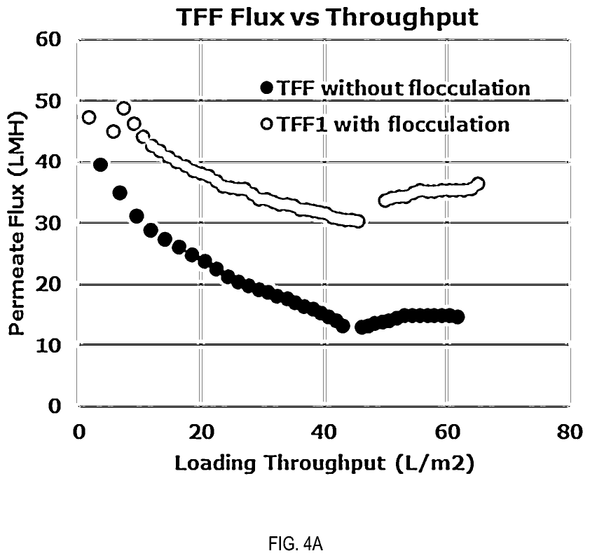

Further analysis was performed using acid glycine-based flocculation at pH 4. The flocculated material was clarified using Clarisolve depth filter. Compared to the HCP level in the post clarification filtrate without flocculation, the post clarification filtrate with flocculation was more than 10 times lower on the HCP level (FIG. 3A). The depth filter performance also was improved after flocculation. Fine particles having an average size of 2 μm in throughput are notable as they readily block depth filters and significantly reduce throughput. As a result of the flocculation process, the fine particles aggregated into larges particles, which improved the depth filter throughput from 31 to 92.5 L/m² with no increase of inlet pressure (FIG. 3B).

Accordingly, glycine can be used to bring the harvest pH down and precipitate impurities. The acid addition rate, agitation in the bioreactor during acid addition, and the scale-up rule were defined to ensure a robust flocculation efficiency in multiple large-scale manufacturing processes.

In some embodiments, implementing a flocculation procedure can improve the performance of one or more purification stages, and/or improve product quality and stability.

### Example 3

To determine the effect of flocculation on downstream aspects of AAV purification, flocculated and non-flocculated material was purified using ultrafiltration/diafiltration, capture chromatography, polishing chromatography.

### Ultrafiltration/Diafiltration

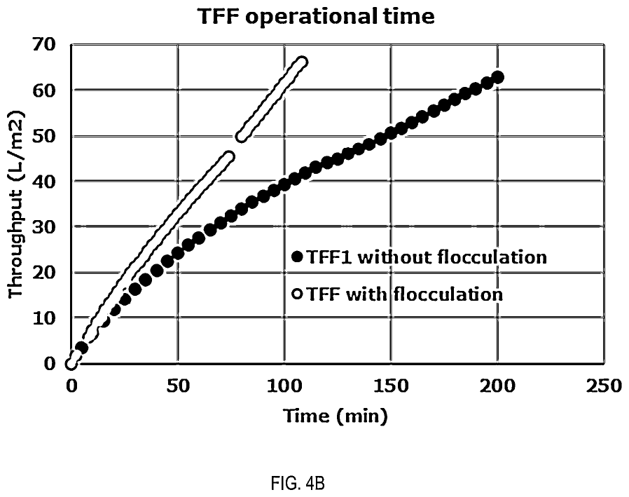

The ultrafiltration/diafiltration performances with and without flocculation are compared in FIGS. 4A and 4B. FIG. 4A shows the UFDF performance with the flocculated material has double flux with no obvious flux decay, compared to the one with non-flocculated material. FIG. 4B shows the operational time with flocculated material is two times faster than the time with non-flocculated material. This can translate to a higher throughput when using flocculated material and potentially saves cost of the goods on UFDF filters. Due to the low impurities (HCL/HC DNA) level in the TFF load after flocculation, the UFDF performed much better with the higher flux, shorter operational time and much clean pool after concentration.

### Capture Chromatography

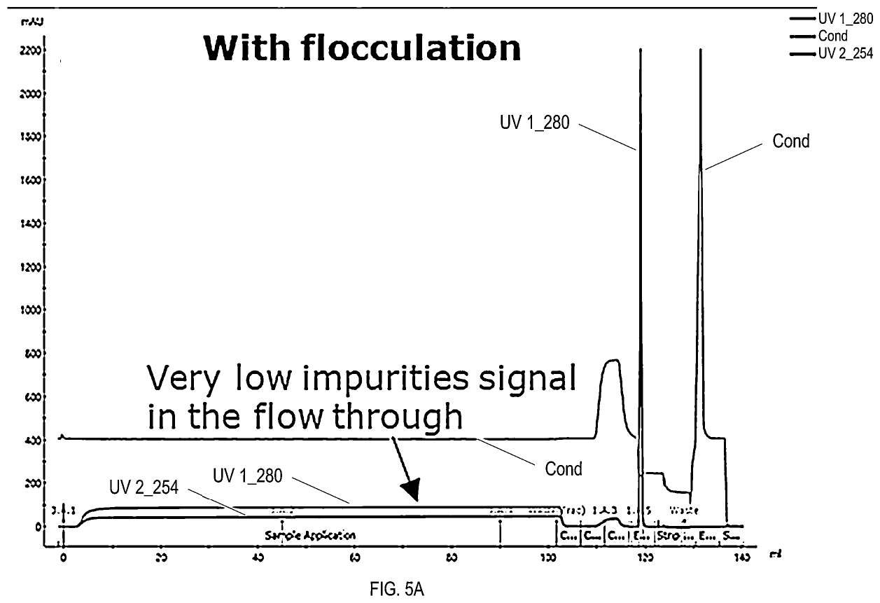

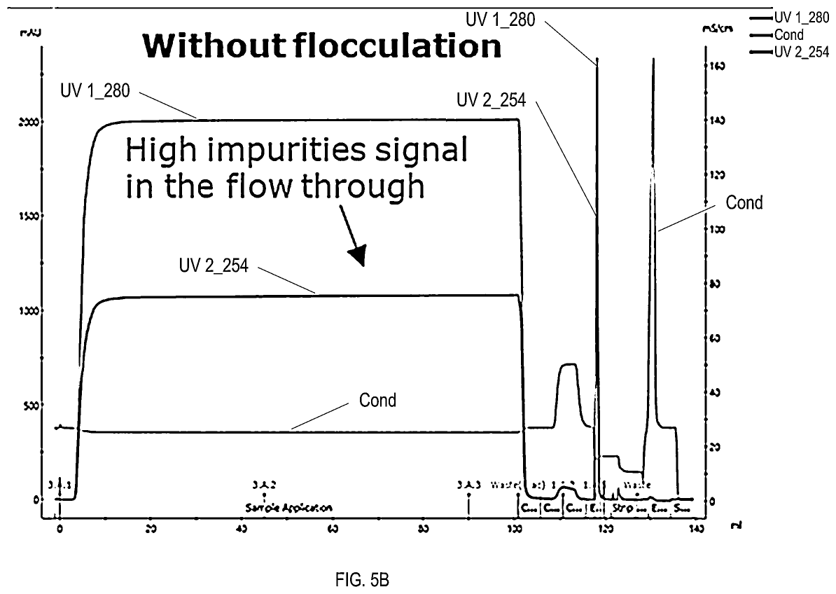

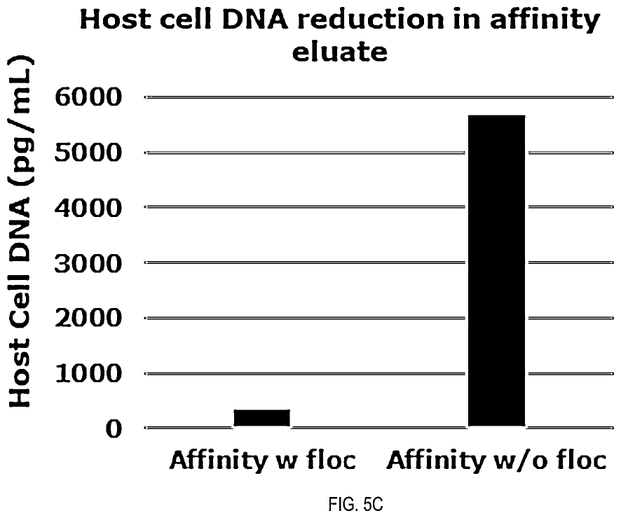

The flocculation impact on capture chromatography (affinity column) is shown in FIGS. 5A, 5B, and 5C. Without flocculation, a very high UV signal in the flow through is observed (FIG. 5B), meaning a high level of impurities in the affinity load. After flocculation, the UV signal in the flow through reduces from 2000 mAU to below 100 mAU (FIG. 5A), showing a very low impurities in the affinity load. The low level of impurities improves the efficiency of column binding and capturing recovery. The host cell DNA in the affinity eluate with and without flocculation is also shown in FIG. 5C. The host cell DNA is more than 10 times lower after flocculation, compared to the affinity eluate without flocculation. This is another evidence that flocculation significantly removes impurities and improves the product quality.

### Polishing Chromatography

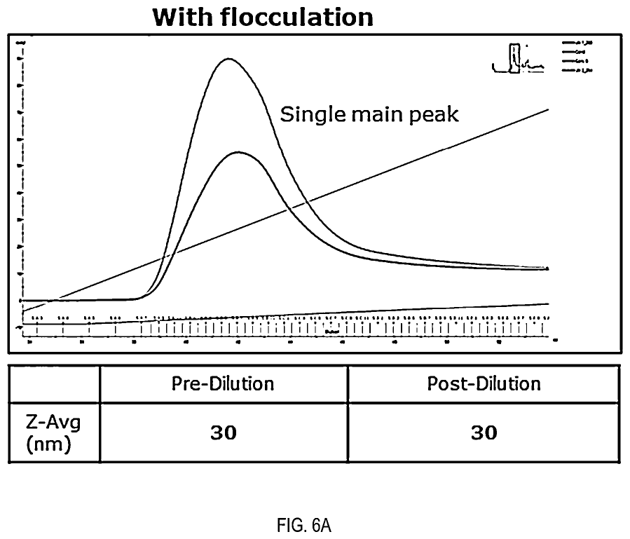

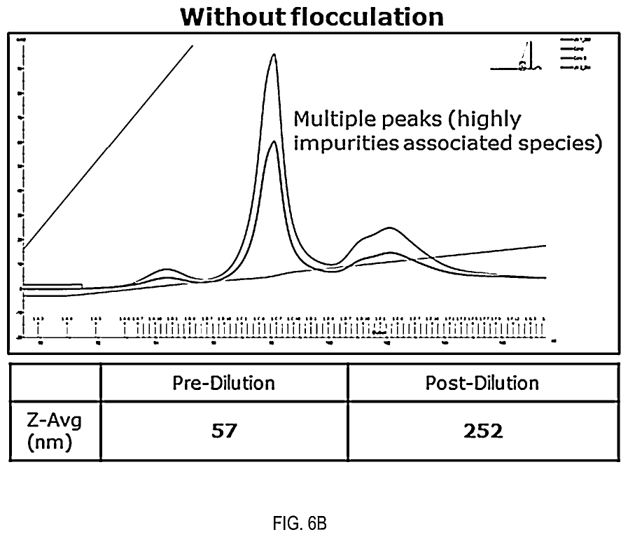

The benefits on polishing chromatography are shown in FIGS. 6A-6B. The material after flocculation shows single peak (FIG. 6A) while the material without flocculation shows multiple peaks with no enrichment. The multiple peak pattern in FIG. 6B means that there might be several impurity-associated species and gives the difficulty to separate full vectors from empty vectors. Impurities can also induce product aggregation and bring the Z-avg larger than 30 nm, especially at low conductivity. The z-avg in the tables show that after dilution, the flocculated material remains 30 nm with superior stability, while the averaged particle size for the non-flocculated material increased significantly.

### Full Vector Enrichment

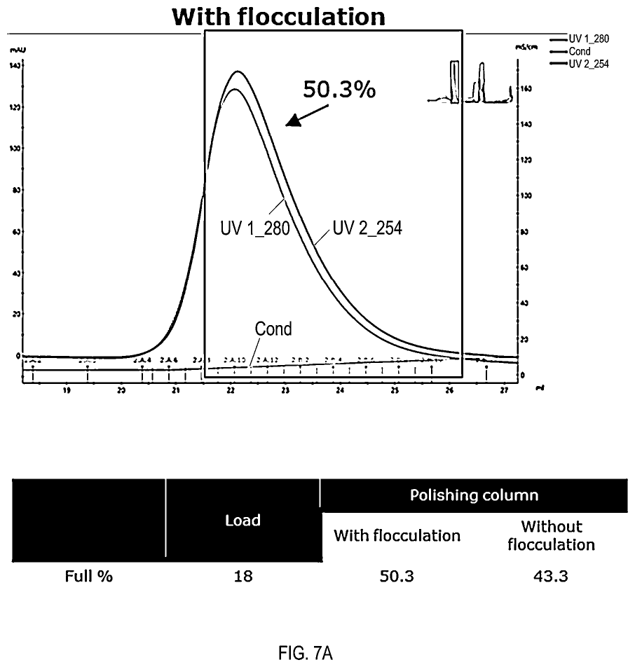

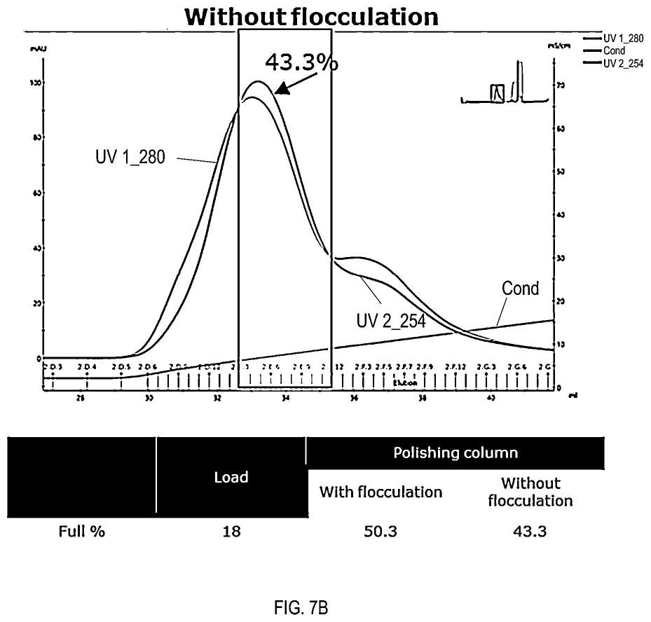

The full vector enrichment after polishing chromatography is shown in FIG. 7A. The material with flocculation shows that full capsid is enriched from 18% to 50.3% on the right side of the main peak. However, the material without flocculation in FIG. 7B shows multiple peaks and the enrichment only occurs in the middle section of the peak. This result demonstrates that, with less impurities associated with the product, it is easier for polishing step to separate full capsid with higher enrichment and higher recovery.

### Product Stability

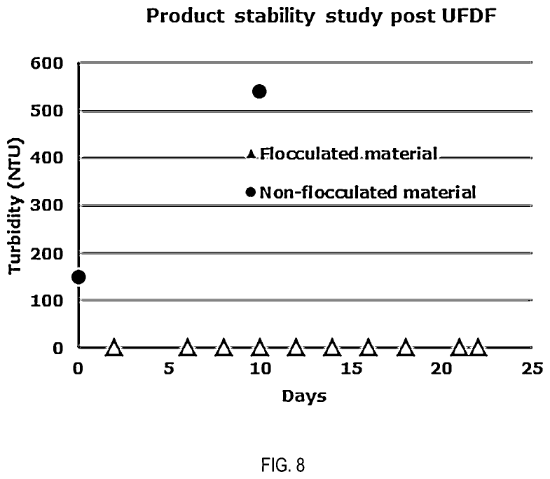

The product stability with and without flocculation is shown in FIG. 8. The material post UFDF is held at room temperature and the turbidity is measured after holding for different periods. Without wishing to be bound by theory, the unstable material will form aggregates and precipitate, resulting in an increase of the turbidity values. The material after flocculation shows stable turbidity profile for over half a month with all the values below 20 NTU, while the turbidity of the material without flocculant treatment increases dramatically from 150 to over 500 NTU within 10 days. As impurities can associate with virus vector product and induce aggregation during hold, the flocculation step can highly stabilize the product.

### Affinity Resin Reuse

Affinity resin is another item that has huge impact to the cost of goods in AAV process development. Recycling affinity resin while having the same purification capacity can significantly reduce the manufacturing cost.

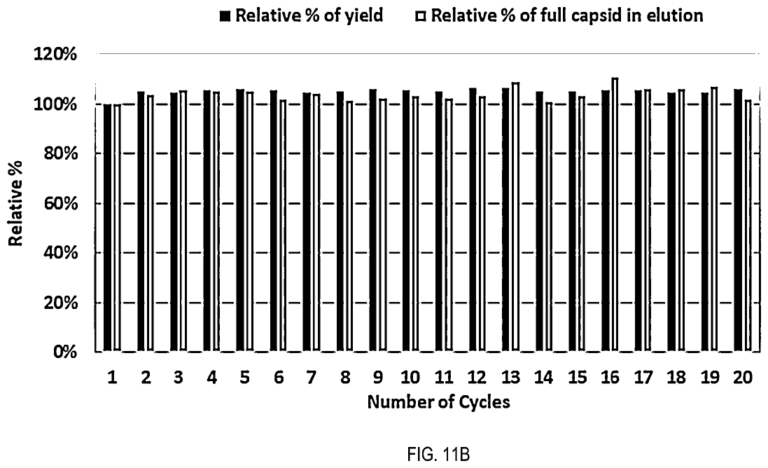

Cell lysate materials with and without flocculation were loaded on to an affinity column. For the non-flocculated material, high content of impurities in the load material has the potential to clog the column or jeopardize the column lifetime. FIGS. 11A and 11B show affinity resin cycling numbers for purifying non-flocculated (A) and flocculated material (B).

As shown in FIG. 11A, the pre-column pressure significantly increased after 5 cycles, which makes the column impossible to reuse for multiple cycles. With flocculation in the harvest step, much cleaner material was loaded on to the affinity chromatography column. The same resin can be used for up to 20 cycles without any impact to yield and product quality in FIG. 11B. The less impurity burden in the affinity load ensures the affinity column has a higher potential for better purification performance with more column cycles and cost saving.

### AAV Aggregation

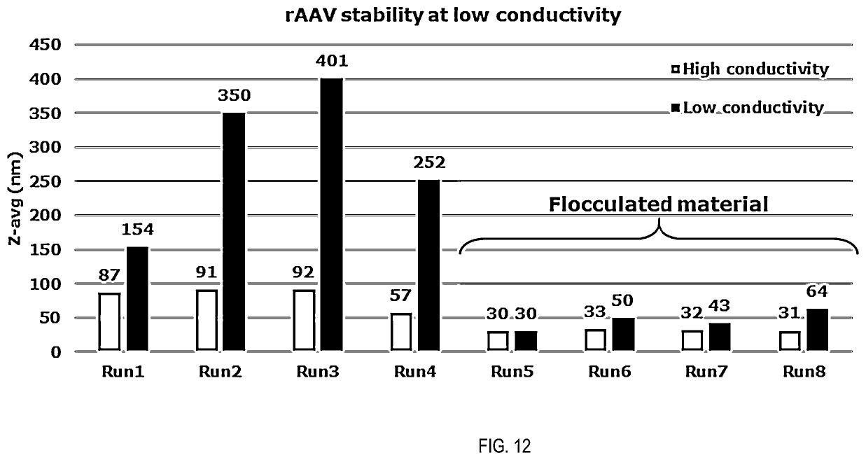

AAV aggregation behavior at low conductivity is the main technical challenge for gene therapy process development. The root cause of the behavior is still unclear, and several hypotheses have been under investigation. One hypothesis that is widely discussed is that the behavior is associated to the impurity profile in the process buffer matrix. Having trace amount of nucleic acid or host cell protein in the process can induce significant product aggregation at low conductivity. Mitigating AAV aggregation at low conductivity has tremendous benefits to the polishing chromatography and improving manufacturing robustness. The AAV aggregation before and after flocculation in the process is illustrated in FIG. 12. Materials in Run 1-4 were not treated by flocculation while materials in Run 5-8 were treated by glycine acid at the harvest process. All eight runs were treated with endonuclease. The rest of the unit operations were kept the same and the high conductivity was measured after affinity chromatography. The aggregation level can be obtained by averaged hydrodynamic diameter (Z-avg). The AAV product aggregated up to 400 nm at low conductivity without flocculation. For Runs 5-8, flocculation step at the harvest controlled the impurity level and provided less impurity amount going into downstream. Due to the removal of impurities, less aggregation was observed at low conductivity indicating e enhanced product stability upon incorporation of the flocculation step as described herein.

### Conclusion

Flocculation using acid precipitation for AAV purification shows significant HCP and HC DNA reduction to the downstream purification. Glycine is used to bring the harvest pH down and precipitate impurities. The acid addition rate, agitation in the bioreactor during acid addition, and the scale-up rule have been defined to ensure a robust flocculation efficiency in multiple large-scale manufacturing. The benefits of implementing flocculation have been shown herein in terms of better product quality and stability, shorter UFDF operational time, higher full virus vector enrichment and yield. This demonstrated method has the potential for other AAV purification process to ensure the process robustness and better product quality performance.

### Example 4

A non-limiting embodiment of the flocculation method described herein was tested on a different rAAV serotype generated from a producer cell line (PCL). The rAAV serotype was different from the rAAV serotype tested in Examples 1-3, which was generated using a triple transfection method.

### —Flocculant Screening

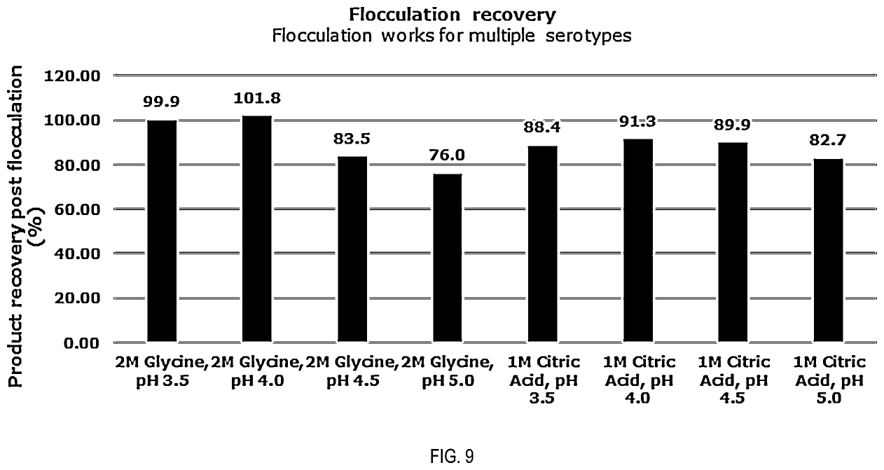

Glycine and citric acid were used at different pHs to treat a cell culture harvest material containing the PCL based rAAV serotype being tested and assess the effect of acid type and pH condition on impurity removal. Results are shown in FIG. 9. Both glycine acid and citrate acid, used as flocculant, have minimal negative impact on the cell culture harvest recovery of the PCL based rAAV serotype, and thus different buffers can be used to lower pH from pH 7.5 or pH 8 down to acidic and pH is the driving force for flocculation. Depending on which acid is used in the later purification step, different acid can be chosen as the flocculant reagent to treat the cell culture harvest, which gives the flexibility of flocculant reagent selectivity.

### Host Cell DNA Removal

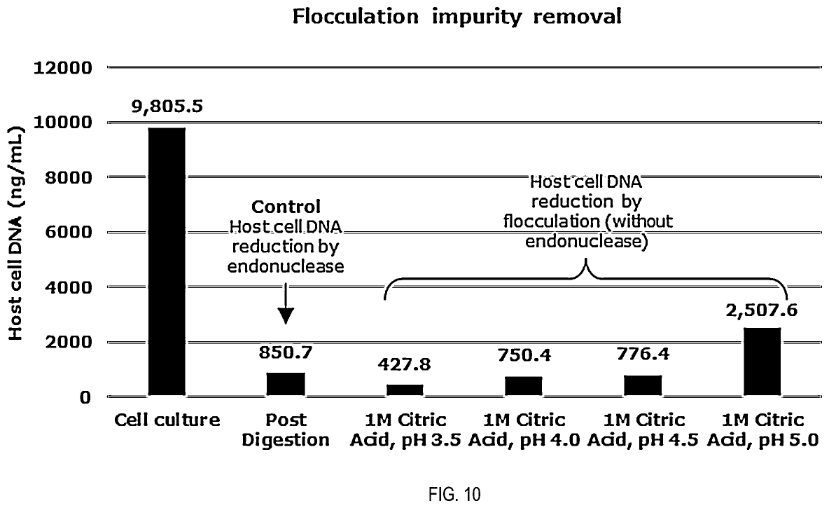

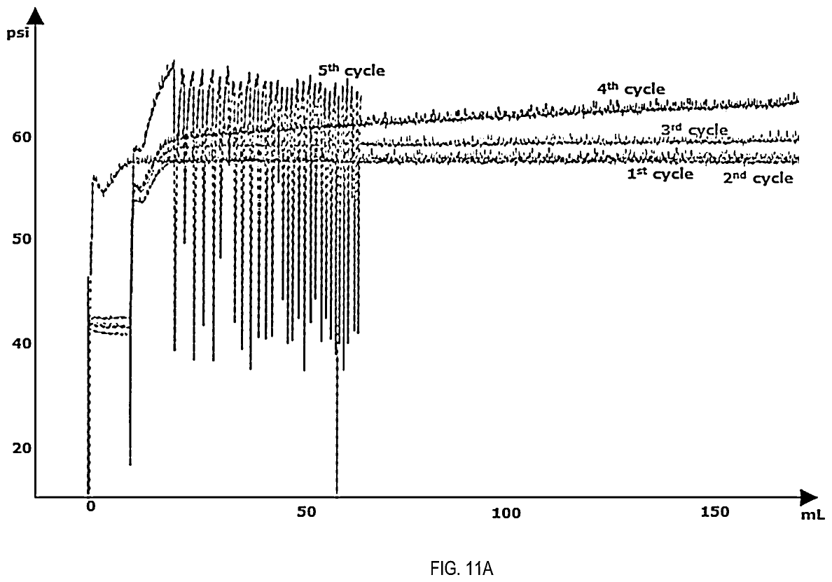

The host cell DNA (HC DNA) concentration was measured under different flocculation condition for the PCL based rAAV serotype. In parallel, endonuclease was used to treat the cell culture material and the HC DNA concentration was quantified as the control. Endonuclease is an expensive enzyme and thus its use has a huge impact on the cost of goods for gene therapy process development. Replacing endonuclease while maintaining the same level of HC DNA reduction is the ideal situation. Flocculation using acid was applied directly to cell culture without endonuclease digestion to investigate the HC DNA reduction level. Compared to endonuclease digestion, the host cell DNA level is significantly reduced from 9806 to below 1000 ng/ml after the acid treatment (FIG. 10). Flocculation method provides a much cleaner upstream material and several benefits to the downstream purification as well as manufacturing robustness. In addition, the endonuclease-free process also contributes towards lowering the manufacturing cost significantly.

### Product Stability During Low pH Hold

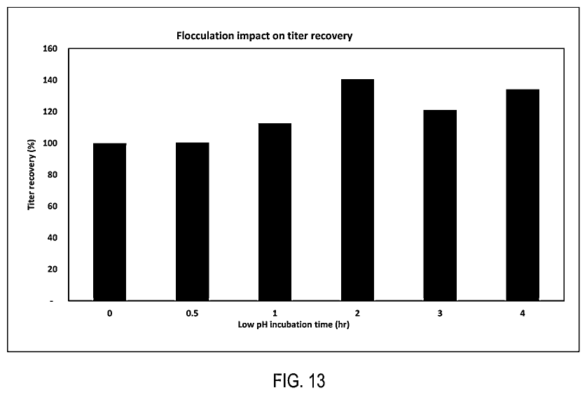

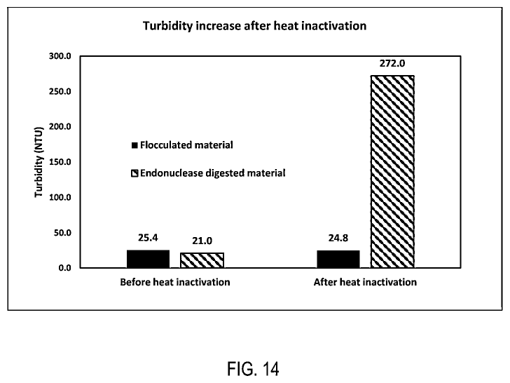

Cell culture harvest containing the PCL based rAAV particles was adjusted to pH 4 using acid buffer for flocculation, and incubated for different periods of time (0.5, 1, 2, 3, 4 hours). The percent recovery results indicated that AAV was stable at pH 4 incubation for at least 4 hours with no titer loss. A slight increase in titer was observed at various time points, ranging from about 10% to 20%. Results are shown in FIG. 13.

### Heat Inactivation

In producer cell line (PCL) platform, Ad5 is introduced in the cell culture harvest and needs to be removed downstream. Heating to a temperature that can inactivate Ad5 while maintaining AAV activity is one main strategy for virus clearance. However, during heat inactivation, impurities (e.g., host cell DNA and proteins) degrade and aggregate, causing a large increase in turbidity. High turbidity poses many challenges to downstream processing, including filtration clogging and product loss. As seen in FIG. 14, flocculated material shows little or no increase in turbidity after heat inactivation, therefore avoiding the difficulties high turbidity imposes.

### EQUIVALENTS

While several inventive embodiments have been described and illustrated herein, those of ordinary skill in the art will readily envision a variety of other means and/or structures for performing the function and/or obtaining the results and/or one or more of the advantages described herein, and each of such variations and/or modifications is deemed to be within the scope of the inventive embodiments described herein. More generally, those skilled in the art will readily appreciate that all parameters, dimensions, materials, and configurations described herein are meant to be exemplary and that the actual parameters, dimensions, materials, and/or configurations will depend upon the specific application or applications for which the inventive teachings is/are used. Those skilled in the art will recognize, or be able to ascertain using no more than routine experimentation, many equivalents to the specific inventive embodiments described herein. It is, therefore, to be understood that the foregoing embodiments are presented by way of example only and that, within the scope of the appended claims and equivalents thereto, inventive embodiments may be practiced otherwise than as specifically described and claimed. Inventive embodiments of the present disclosure are directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the inventive scope of the present disclosure.

All definitions, as defined and used herein, should be understood to control over dictionary definitions, definitions in documents incorporated by reference, and/or ordinary meanings of the defined terms.

All references, patents and patent applications disclosed herein are incorporated by reference with respect to the subject matter for which each is cited, which in some cases may encompass the entirety of the document.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e., “one or the other but not both”) when preceded by terms of exclusivity, such as “either,” “one of,” “only one of,” or “exactly one of.” “Consisting essentially of,” when used in the claims, shall have its ordinary meaning as used in the field of patent law.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

It should also be understood that, unless clearly indicated to the contrary, in any methods claimed herein that include more than one step or act, the order of the steps or acts of the method is not necessarily limited to the order in which the steps or acts of the method are recited.

In the claims, as well as in the specification above, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively, as set forth in the United States Patent Office Manual of Patent Examining Procedures, Section 2111.03. It should be appreciated that embodiments described in this document using an open-ended transitional phrase (e.g., “comprising”) are also contemplated, in alternative embodiments, as “consisting of” and “consisting essentially of” the feature described by the open-ended transitional phrase. For example, if the disclosure describes “a composition comprising A and B”, the disclosure also contemplates the alternative embodiments “a composition consisting of A and B” and “a composition consisting essentially of A and B”.

1. A method of purifying recombinant adeno-associated virus (rAAV) particles from a cell culture comprising the rAAV particles, the method comprising:
a) contacting an rAAV preparation obtained from a cell culture with an acid solution under conditions sufficient to promote flocculation of cellular material that is present in the rAAV preparation; and
b) separating rAAV particles from flocculated cellular material,
optionally wherein the acid solution comprises an acid glycine solution.

2. The method of claim 1, wherein the rAAV preparation is a cell culture harvest comprising rAAV particles.

3. (canceled)

4. The method of claim 1, further comprising contacting the rAAV preparation with a nuclease and/or subjecting the preparation to cell lysis.

5. The method of claim 1, wherein the pH of the acid solution is below 4.

6-7. (canceled)

8. The method of claim 1, wherein the acid solution is added to the rAAV preparation at a volume of 5-10%.

9. (canceled)

10. The method of claim 1, wherein the acid solution is added to the rAAV preparation within a period of 10 minutes.

11. (canceled)

12. The method of claim 1, wherein the rAAV preparation is mixed with the acid solution using an agitation speed is 30-150 RPM.

13. (canceled)

14. The method of claim 1, wherein the volume of the rAAV preparation is 2-500L.

15-18. (canceled)

19. The method of claim 1, wherein the mixture of the rAAV preparation and the acid solution of a) has a pH of 3-5.

20. (canceled)

21. The method of claim 1, wherein the mixture of the rAAV preparation and the acid solution of a) is held static in a vessel for 10-60 minutes to promote flocculation of the cellular material.

22-23. (canceled)

24. The method of claim 1, wherein the mixture of the rAAV preparation and the acid solution from a) produces a flocculated material and the flocculated material is resuspended prior to separating AAV particles from flocculated cellular material.

25. The method of claim 1, wherein the mixture of the rAAV preparation and the acid solution of a) is clarified.

26-28. (canceled)

29. The method of claim 1, wherein the rAAV particles are recombinant AAV1, AAV2, AAV3, AAV4, AAV5, AAV6, AAV7, AAV8, AAV9, AAV10, AAV11, or AAV12 particles, derivatives and/or in combination thereof, or wherein the rAAV particles comprise a hybrid capsid.

30-31. (canceled)

32. The method of claim 1, wherein a plurality of the rAAV particles encapsulate a recombinant nucleic acid comprising a gene of interest flanked by AAV ITRs.

33-34. (canceled)

35. A composition comprising rAAV particles produced by the method of claim 1.

36. A method comprising administering the composition of claim 35 to a subject.

37. A method of purifying rAAV particles from a cell culture comprising the rAAV particles, the method comprising:
a) contacting an rAAV preparation obtained from a cell culture with a flocculation agent under conditions sufficient to promote flocculation of cellular material that is present in the rAAV preparation; and
b) separating rAAV particles from flocculated cellular material.

38. The method of claim 37, wherein the flocculation agent is a pH-reducing agent or a cationic polymer.

39-40. (canceled)

41. A method of purifying rAAV particles from a cell culture comprising the rAAV particles, the method comprising:
a) contacting an rAAV preparation obtained from a cell culture with a flocculation agent under conditions sufficient to promote flocculation of cellular material that is present in the rAAV preparation; and
b) separating rAAV particles from flocculated cellular material, wherein the method does not comprise the steps of (i) contacting the rAAV preparation with an endonuclease and/or (ii) subjecting the rAAV preparation to a cell lysis.

42-43. (canceled)

44. The method of claim 1, wherein the cell culture comprises a density of 0.5-12×10⁶ cells/mL

45-46. (canceled)
